(19) United States
(12) Patent Application Publication
(10) Pub. No.: US 2024/0355411 A1
(43) Pub. Date: Oct. 24, 2024

(54) DECODING SURFACE FINGERPRINTS FOR PROTEIN-LIGAND INTERACTIONS

(71) Applicants: ÉCOLE POLYTECHNIQUE FÊDÉRALE DE LAUSANNE (EPFL), Lausanne (CH); IMPERIAL COLLEGE INOVATIONS LIMITED, London (GB)

(72) Inventors: Bruno Emanuel Ferreira De Sousa Correia, Saint-Saphorin-sur-Morges (CH); Michael Bronstein, London (GB); llia Igashov, Écublens (CH)

(21) Appl. No.: 18/621,396

(22) Filed: Mar. 29, 2024

Related U.S. Application Data
(60) Provisional application No. 63/455,819, filed on Mar. 30, 2023.

(51) Int. Cl. G16B 15/30; G16B 40/20

(52) U.S. Cl. CPC G16B 15/30; G16B 40/20

(57) ABSTRACT

Small molecules have been the preferred modality for drug development and therapeutic interventions. This molecular format presents a number of advantages, e.g. long half-lives and cell permeability, making it possible to access a wide range of therapeutic targets. However, finding small molecules that engage “hard-to-drug” protein targets specifically and potently remains an arduous process, requiring experimental screening of extensive compound libraries to identify candidate leads. The search continues with further optimization of compound leads to meet the required potency and toxicity thresholds for clinical applications. Here, we propose a new computational workflow for high-throughput fragment-based screening and binding affinity prediction where we leverage the available protein-ligand complex structures using a state-of-the-art protein surface embedding framework (dMaSIF). We developed a tool capable of finding suitable ligands and fragments for a given protein pocket solely based on protein surface descriptors, that capture chemical and geometric features of the target pocket. The identified fragments can be further combined into novel ligands. Using the structural data, our ligand discovery pipeline learns the signatures of interactions between surface patches and small pharmacophores. On a query target pocket, the algorithm matches known target pockets and returns either potential ligands or identifies multiple ligand fragments in the binding site. Our binding affinity predictor is capable of predicting the affinity of a given protein-ligand pair, requiring only limited information about the ligand pose. This enables screening without the costly step of first docking candidate molecules. Our framework will facilitate the design of ligands based on the target's surface information. It may significantly reduce the experimental screening load and ultimately reveal novel chemical compounds for targeting challenging proteins.

### CROSS REFERENCE TO RELATED APPLICATIONS

This application claims the benefit of U.S. Provisional Application No. 63/455,819 (filed on Mar. 30, 2023), the entirety of which is incorporated by reference herein. This application shares at least one common inventors with those individuals named as authors of the following publication: Igashov et al., “DECODING SURFACE FINGERPRINTS FOR PROTEINLIGAND INTERACTIONS,” MLDD Workshop, ICLR (Mar. 31, 2022). The entirety of the foregoing publication is incorporated by reference herein.

### BACKGROUND

### 1 Introduction

Small molecule drug discovery is an arduous and resource-intensive process, characterized by declining productivity and high attrition rates. Computational design and, in particular, computational structure-based drug design (SBDD)—where the 3D structure of the target protein is known—of small molecules has been a successful strategy in developing novel therapies. Typical computational SBDD workflows are based on virtual screening of large libraries of drug-like compounds, which are docked and scored by scoring functions to determine ideal geometries and complementarity. Various deep learning approaches have been applied to components of SBDD with promising results. However, rational de novo design has been relatively less studied by the machine learning community.

Fragment-based drug discovery (FBDD) has been a successful paradigm in early-stage drug development. The majority of the current workflows involve screening libraries of low molecular weight fragments against macromolecular targets of interest. Fragments can occupy multiple binding locations on the target providing starting points for developing candidate compounds through growing, linking or merging fragments. By incorporating structural knowledge of the target, a bottom-up approach like FBDD can result in reduced cost compared to high-throughput screening workflows. Furthermore, by designing candidate molecules around constituent favorable interactions, improved ligand efficiency can be achieved. However, fragments typically bind with much lower affinities than drug-like molecules making their identification and structural characterisation difficult.

Geometric deep learning, which encompasses deep neural networks operating on non-Euclidean domains, has shown considerable benefits in representation learning on proteins and other molecular structures, particularly in the context of drug discovery.

### 1.1 Related Work

### Pocket-Centered Ligand Screening

Binding pocket similarity plays an important role in drug discovery. Incorporating structural knowledge about pockets of interest enables efficient search of relevant binding molecules and provides new means for exploring molecular space. Provided that a ligand can be recognized by different residues with different interaction types, finding a suitable pocket representation is the most crucial and highly challenging part of the task. Various pocket representations have been explored, including pharmacophore and deep learning-based fingerprints as well as geometric and chemo-physical descriptors. Recently, pocket-centered methods for conditional molecule generation have been proposed. For example, a geometric deep learning method for predicting the receptor binding location and the ligand's bound pose and orientation was developed.

### Fragment-Based Drug Discovery

In a standard setting, screening methods are fundamentally limited by the diversity of the underlying compound libraries. FBDD operates on small labile molecules (typically with molecular weight <300 Da) to identify low potency, high-quality leads, which are then matured into more potent, drug-like compounds. Millions of compounds such as rings, linkers and scaffolds are available in databases such as ZINC. However, identifying and filtering relevant fragments from such a database is a challenging problem. Instead, one can split molecules of interest into smaller compounds based on various chemical and geometric assumptions to generate a relevant fragment database. Another FBDD-specific problem is constructing a ligand from the found fragments. Various linking and scaffold hopping methods have been developed to address this task. Recently, deep generative models have been proposed or adopted for these purposes. Notably, a structure-based method was developed for generating drug-like ligands from molecular fragments via a sequential VAE-based approach, conditioning each step of the generative process on a graph representation of the binding pocket.

### Binding Affinity Prediction

Predicting the affinity of a given protein-ligand interaction is an important component of virtual screening pipelines. Traditionally, methods for predicting binding affinity have been based on physics-based free energy calculations which do not account for entropic contributions. Machine learning approaches based on hand-crafted features, which do not impose a pre-defined functional form, were shown to improve performance by learning from the experimental data. Several types of deep learning models have been applied to this problem, including 3D-CNNs, GNNs, and surface-based models. However, the majority of existing ML-based approaches heavily rely on co-crystallized structures of complexes which are hard and not always possible to obtain. Often, docking algorithms are used which typically result in noisier and more error-prone poses that prove more challenging to accurately predict binding affinity from. Furthermore, these approaches are insensitive to scenarios where multiple binding mechanisms and poses contribute to the overall affinity.

### Protein Surface Representation

Structure-based encoders of protein molecular surfaces have been successfully applied to predicting protein-protein interaction sites and identifying potential binding partners in protein docking. Molecular surface interaction fingerprinting (MaSIF) pioneered learning geometric surface descriptors for solving protein interaction-related tasks. MaSIF computes geometric and chemical features on a triangulated surface mesh and aggregates local information using a geometric deep learning architecture based on geodesic convolution operators. Differential MaSIF (dMaSIF) as described by Freyr Sverrisson, Jean Feydy, Bruno E Correia, and Michael M Bronstein in Fast end-to-end learning on protein surfaces as published in Proceedings of the IEEE/CVF Conference on Computer Vision and Pattern Recognition, pp. 15272-15281, 2021, (herein after Sverrisson 2021), which is incorporated by reference herein in its entirety, is a derived method that sidesteps the expensive mesh generation and feature pre-computation steps and creates a lightweight point cloud representation of the molecular surface solely from raw atom coordinates and types. Feature vectors at each surface point are updated by applying approximate geodesic convolutions resulting in the final embedding vectors that can be further used in various downstream tasks. In this work, we employ these vectors to compare protein binding pockets and to predict protein-ligand binding affinity. FIG. 1 illustrates the workflow of dMaSIF.

### SUMMARY

Described herein is a novel ligand and fragment searching methodology based on geometric deep learning applied to surface representations of binding pockets. The effectiveness of this method is demonstrated on a subset of the PDBbind dataset documented by Renxiao Wang, Xueliang Fang, Yipin Lu, and Shaomeng Wang in their paper The PDBbind database: collection of binding affinities for protein-ligand complexes with known three-dimensional structures as published in Journal of Medicinal Chemistry, 47 (12): 2977-2980 May 2004 (herein after Wang 2004), which is incorporated by reference herein in its entirety. The demonstration shows that the proposed approach performs on par with the state of the art and outperforms a recent deep learning-based method. A binding affinity prediction model competitive with the current state of the art is also described herein. This method requires limited knowledge of the binding pose, accelerating virtual screening workflows by circumventing docking and screening of compounds and targets for which co-crystallised structures are not available.

### BRIEF DESCRIPTION OF THE DRAWINGS

FIG. 1 shows a surface encoding workflow of a dMaSIF, a library construction process, and a ligand search pipeline.

FIG. 2A shows Multidimensional scaling applied to pairwise similarities of pockets corresponding to five different ligands, where each point represents a single pocket colored by the corresponding ligand type.

FIG. 2B shows a Fragment search examples, where for each ligand, the most suitable found and aligned fragments from top-10 are shown and where thin lines represent parts of ligands that are not covered by fragments.

FIG. 3 shows Distribution of RMSD values over pocket pairs before and after alignment.

FIG. 4 shows examples of fragments retrieved using BRICS.

### DETAILED DESCRIPTION

### 2 Methods

### 2.1 Ligand Search

A typical pocket-centered drug discovery workflow involves screening libraries containing millions of molecules to identify an initial set of candidates. To ensure effectiveness, both a suitable pocket description and appropriate search procedure are required. In this section, a novel pocket-centered ligand search pipeline is introduced. A library of available (known) ligand-pocket pairs are constructed and are queried by new (query) pockets to identify candidate ligands that are most likely to bind to the input pockets.

### Dataset Preparation

A subset of PDBbind v2020 database, documented in Wang 2004, is considered by combining the general (n=14, 127) and refined (n=5, 316) sets and selecting only complexes with ligands that are not amino acids, which are subsequently parsed by RDKit, have QED>0.3, and are present in more than 10 complexes. 488 complexes are obtained and the structures are protonated with Reduce. To construct the query (n=130) and library (n=358) datasets, sequence similarity clusters are computed for all proteins using CD-HIT with a similarity threshold of 0.9. For each ligand, we split the set of the corresponding complexes in query and library sets in proportion 30/70, such that clusters assigned to each set do not overlap.

### Protein Pockets

To retrieve the protein pocket, a surface of the protein is first generated and embeddings are computed for each point on the surface using dMaSIF. For each ligand atom, the closest point on the protein surface is selected. For each selected point, its r-neighborhood, a set of points within Euclidean distance r, is considered. Different values for r (see Appendix A) are considered, and the best performance is achieved with r=2 Å. The resulting pocket embedding is generated as a union of r-neighborhoods of all selected points.

### Workflow

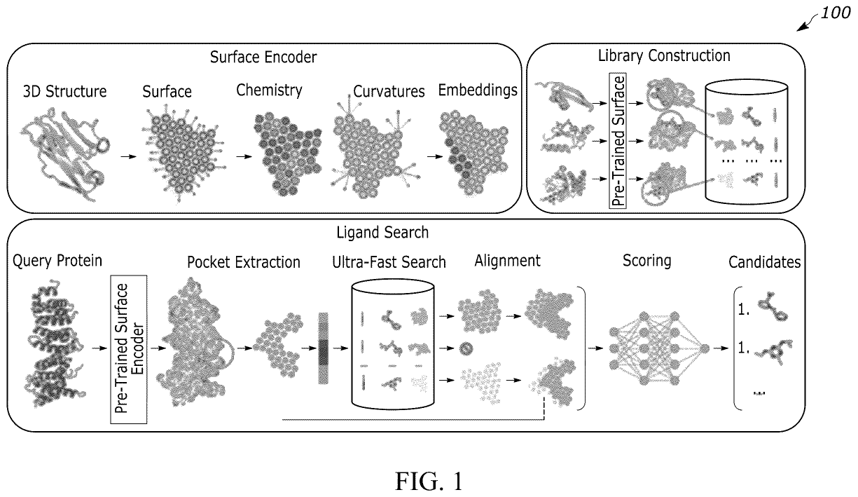

For each pocket in the query set, the most similar binding pockets in the library are searched for and the corresponding ligands are output as candidates for the query pocket. The general scheme of the workflow 100 is represented in FIG. 1 and consists of three main steps: shortlisting, alignment and scoring. First, an ultra-fast search is performed over the whole library in order to shortlist candidate ligands. The purpose of this step is to filter out irrelevant ligands and hence reduce the computational complexity of the subsequent steps. For each query pocket, the top-50 candidates are selected based on similarity between global pocket embeddings. As shown in Appendix A, two similarity functions are considered: Euclidean distance and dot product. The best performance is achieved using Euclidean distance. Global pocket embeddings are created by averaging the embedding vectors of all points in the pocket. An alternative approach was to consider an embedding vector of a single point, e.g. of the one closest to the center of the pocket. However, as shown in Appendix A, better results were achieved with averaging. In the second step, pockets of shortlisted candidates are aligned with the query pocket. Two methods are considered: Random Sample Consensus (RANSAC) followed by point-to-point Iterative Closest Point (ICP), and the optimization-based alignment approach described in Section 2.2 below. Once the shortlisted pockets are aligned with the query pocket, pairs of pockets are scored using a pre-trained neural network (described in Section 2.3).

### 2.2 Optimization-Based Alignment

Consider two point clouds: a source point cloud with M points described by coordinates {rⱼ}ⱼ₌₁^{M}, normal vectors {nⱼ}ⱼ₌₁^{M}, and embedding vectors {fⱼ}ⱼ₌₁^{M}, and a target point cloud with N points described by coordinates {rᵢ}ᵢ₌₁^{N}, normal vectors {nᵢ}ᵢ₌₁^{N}, and embedding vectors {fᵢ}ᵢ₌₁^{N}.

We represent normal vectors and embeddings of the target point cloud as continuous vector fields n(r)=Σᵢ₌₁^{N}nᵢgᵢ(r) and f(r)=Σᵢ₌₁^{N}fᵢgᵢ(r) correspondingly in R³ by smoothing the discrete points of the target point cloud via Gaussian kernels

$${ℊ}_{i} ( r ) = \frac{1}{\sqrt{2 {πσ}^{2}}} exp ( \frac{{‖ r - {r}_{i} ‖}_{2}}{2 {σ}^{2}} ) , i ∈ \{ 1 , … , N \} , σ ∈ ℝ . \qquad ( 1 )$$

The task is to find the optimal orientation of the source point cloud with respect to the vector fields f(r) and n(r). The best orientation of the source point cloud is the orientation that maximizes two terms. The first term is similarity S between embedding vectors of the source point cloud and values of the field f(r) in coordinates that correspond to the positions of points of the source point cloud. Similarity can be expressed as Euclidean distance, dot product, or cosine between two vectors. The second term is dot product between normal vectors of the source point cloud and values of the field n(r) in coordinates that correspond to the positions of points of the source point cloud. Orientation of the source point cloud is parameterized by rotation matrix R∈M₃(R), R^{T}R=I, det R=1, and translation vector t∈R³. The objective function can be written as follows,

$$ℒ ( R , t ) = \frac{1}{M} {\sum }_{j = 1}^{M} [ β S ( {f}_{j} , f ( {Rr}_{j} + t ) ) + ( 1 - β ) {( {Rn}_{j} )}^{T} n ( {Rr}_{j} + t ) ] , \qquad ( 2 )$$

where β∈[0, 1] is a parameter that controls the contribution of each term in the final objective value. Using a 6D-parameterization R=R(u, v) of rotation matrix R with u∈R³, v∈R³, as described by Yi Zhou, Connelly Barnes, Jingwan Lu, Jimei Yang, and Hao Li in On the continuity of rotation representations in neural networks published in Proceedings of the IEEE/CVF Conference on Computer Vision and Pattern Recognition, pp. 5745-5753, 2019, the optimization problem can be written as max_{u,v,t∈R}3 L (R(u, v), t). To find the optimal rotation and translation parameters, gradient descent is performed in R³×R³×R³ implemented in PyTorch using Adam optimizer.

### 2.3 Scoring Neural Network

For the final ranking after pocket alignment, a neural network is trained to distinguish between good—where pockets correspond to the same ligand and are properly aligned—and bad matches. The training dataset is crucial to the method's success as it implicitly defines a notion of quality that is subsequently used to score shortlisted pairs of pockets.

### Dataset

Data is prepared in a similar procedure to Section 2.1. However, here ligands are selected that are represented in fewer than ten complexes. This means that complexes used for training are not used in the ligand search experiments. To create positive training examples, pairs of pockets are selected that bind to the same ligands but originate from complexes with less than 90% sequence identity. These pockets are aligned based on ligand atom coordinates using rigid-body SVD-based alignment. To create negative examples pairs of pockets are randomly selected that correspond to different ligands and match their centers of mass. The positive and negative example pairs are randomly split into training (n=2, 562) and validation (n=654) sets.

### Input Features

For a given pair of pockets, their point-wise features that are input to the network are first computed. For each point in the source pocket the closest point in the target pocket is found, and for each of the resulting point pairs, three values are computed: inverted distance between two points, dot product of normal vectors, and dot product of embedding vectors.

### Architecture

The scoring model consists of two symmetric blocks with global average pooling between them. The first block takes three-dimensional feature vectors for each point and projects them to the 256-dimensional hidden vector via three fully-connected layers followed by batch normalization layers and ReLU activations. Global pooling averages hidden vectors over all points in the source pocket resulting in a single 256-dimensional vector which is processed by the final block. This block includes a sequence of three fully connected layers with ReLU activations followed by softmax. For symmetry, each pocket pair is processed twice: with the original order of source-target pockets and with the swapped order. Final predictions are averaged.

### 2.4 Fragment Search

Fragment-based search has a notable advantage compared to the ligand search. Although the number of unique available ligands is usually orders of magnitude larger than the number of unique constituent fragments, the latter can be considered as building blocks of ligands. Therefore, the fragment-based method becomes less dependent on the available data and allows exploration of ligand space by combining available fragments. The modified search approach that operates on fragments instead of entire ligands is described below.

### Fragment Generation

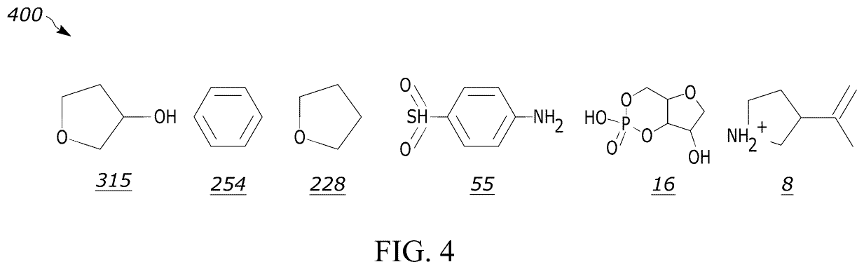

To decompose ligands into fragments, BRICS as described by Jörg Degen, Christof Wegscheid-Gerlach, Andrea Zaliani, and Matthias Rarey in On the art of compiling and using ‘drug-like’ chemical fragment spaces as published in ChemMedChem: Chemistry Enabling Drug Discovery, 3 (10): 1503-1507, 2008, which is incorporated by reference herein in its entirety, is used. BRICS simultaneously breaks retrosynthetically relevant bonds and filters unwanted chemical motifs and small terminal fragments. For each ligand in the dataset described in Section 2.1, its BRICS fragments are retrieved, open exits are remove, and fragments with molecular mass >300 Da are filtered out, the resulting set is duplicated based on Tanimoto distance, and every resulting compound is matched with other ligands in order to get all its occurrences. In total, 36 unique fragments are gathered. Exemplary fragments 400 along with their occurrences are provided in FIG. 4. Mapping unique fragments back to ligands in which they occur, the query (440 entries) and library (1,434 entries) datasets for fragment search are obtained.

### Workflow

The fragment search process is very similar to the ligand-based algorithm described in Section 2.1 and fragment pockets are constructed in the same way. However, as fragments are smaller, the resulting point clouds constructed for fragments describe smaller regions of ligand binding pockets. For clarity, these regions are referred to as fragment patches. The main difference between ligand and fragment search workflows lies in the first step, ultra-fast search. In ligand search, the top-ranked candidates in the library are returned. In fragment search, for each fragment in the library (that can have more than one corresponding patch), a single patch is selected that has the highest similarity score among all patches corresponding to a given fragment. This allows the diversity of candidates in the subsequent steps of the pipeline to be increased.

### 2.5 Binding Affinity Prediction

### Dataset Preparation

PDBbind v2016 is used. PDBbind v2016 is a dataset of protein structures co-crystallised with ligands and their associated binding affinity and is described by Zhihai Liu, Minyi Su, Li Han, Jie Liu, Qifan Yang, Yan Li, and Renxiao Wang in Forging the basis for developing protein-ligand interaction scoring functions as published in Accounts of Chemical Research, 50 (2): 302-309, February 2017, which is incorporated by reference herein in its entirety. Structures are protonated with Reduce and only atoms belonging to the polypeptide chain(s) are extracted. To enable comparison with existing methods, the training set is constructed from the refined set, from which we randomly sample 10% for the validation set. Models are evaluated on the CASF 2016 core set described by Minyi Su, Qifan Yang, Yu Du, Guoqin Feng, Zhihai Liu, Yan Li, and Renxiao Wang in Comparative assessment of scoring functions: The CASF-2016 update as published in Journal of Chemical Information and Modeling, 59 (2): 895-913, November 2018 (which is removed from the training data). Binding pocket embeddings are extracted as described in Section 2.1 using a much larger neighbourhood radius of r=12 Å to reduce the sensitivity to pose. Ligands are represented as molecular graphs. Edges correspond to chemical bonds (single, double, triple, aromatic or ring) and node features include one-hot encoded atom types, degree, valence, hybridization state, number of radical electrons, formal charge, and aromaticity. Crucially, no positional information in the ligand representation is used.

### Architecture

A model is trained using a dMaSIF-based encoder for the protein pocket. Importantly, the encoder receives the whole protein surface. After encoding, pocket point embeddings are extracted and aggregated into a global embedding vector by taking the element-wise max of individual point embeddings as we found this resulted in better performance than sum or average pooling. To focus on understanding the effectiveness of surface-based descriptors, a simple GCN is used as the ligand encoder and aggregate node embeddings via sum pooling to obtain the graph representation. Ligand and pocket embeddings are concatenated and used as input for an MLP decoder to predict the binding affinity values. Hyperparameters are provided in Table S11 below.

### 3 Results

### 3.1 Ligand Search

In this experiment, the ability of the proposed multi-staged search algorithm to output relevant ligands to the query pockets was evaluated. The approach described herein was compared with three state-of-the-art methods for pocket-centered ligand screening, ProBiS, KRIPO, and DeeplyTough. ProBiS detects structurally similar sites on protein surfaces by local surface structure alignment using an efficient maximum clique algorithm. KRIPO is a method for quantifying the similarities of binding site subpockets based on pharmacophore fingerprints. DeeplyTough is a convolutional neural network that encodes a three-dimensional representation of protein pockets into descriptor vectors that are further compared using Euclidean distance. KRIPO failed on 6 query pockets, hence all the results discussed below were obtained on 124 query pockets successfully processed by all the methods.

Table 1 reports the fractions of pockets from the query set for which the methods returned correct ligands in top-1, top-5, top-10, top-20, and top-50. The best performing method operates on pockets built with neighborhood radius r=2 Å and using a dMaSIF-search model trained (for 33 epochs) with the standard parameters except subsampling (set to 150), resolution (set to 0.7 Å), and embedding size (set to 16). Metrics computed after the first and last stages of the search process are reported. Namely, metrics were computed for lists of candidates ranked by global search score and neural network score. Results of pipelines that use RANSAC and the optimization method described herein are provided for alignment.

TABLE 1
Ligand search results.

| Method | Top-1 | Top-5 | Top-10 | Top-20 | Top-50 |
|---|---|---|---|---|---|
| ProBiS (Konc & Janez “ic”, 2010) | 0.581 | 0.718 | 0.750 | 0.806 | 0.863 |
| KRIPO (Wood et al., 2012) | 0.573 | 0.726 | 0.798 | 0.863 | 0.935 |
| DeeplyTough (Simonovsky & Meyers, 2020) | 0.331 | 0.597 | 0.710 | 0.758 | 0.903 |
| Ours: search | 0.387 | 0.516 | 0.629 | 0.734 | 0.871 |
| Ours: search + ransac + score | 0.516 | 0.661 | 0.742 | 0.823 | 0.871 |
| Ours: search + optim + score | 0.516 | 0.637 | 0.710 | 0.766 | 0.871 |

The full-scale search pipeline performs on par with state-of-the-art methods (Table 1). Notably, the first stage of the search process, ultra-fast global search, returns relevant ligands within the top-50 candidates in 87% of cases, making it appropriate for the initial shortlisting of candidates prior to fine-grained scoring.

### Pocket Clustering

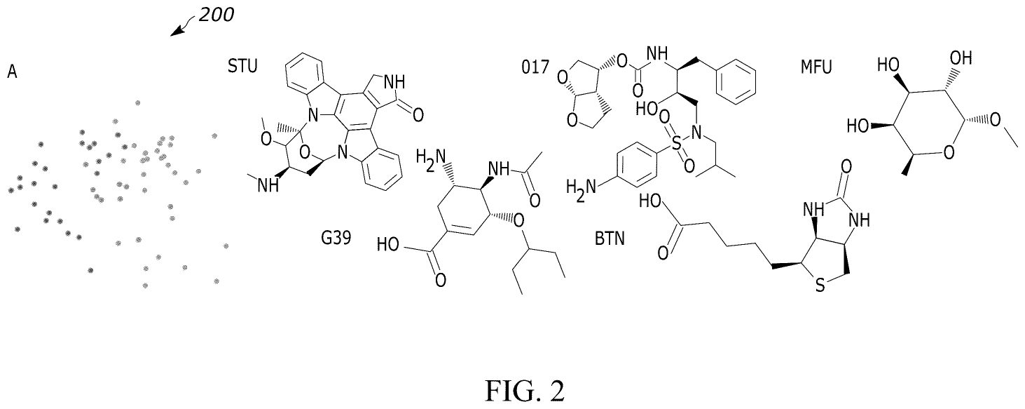

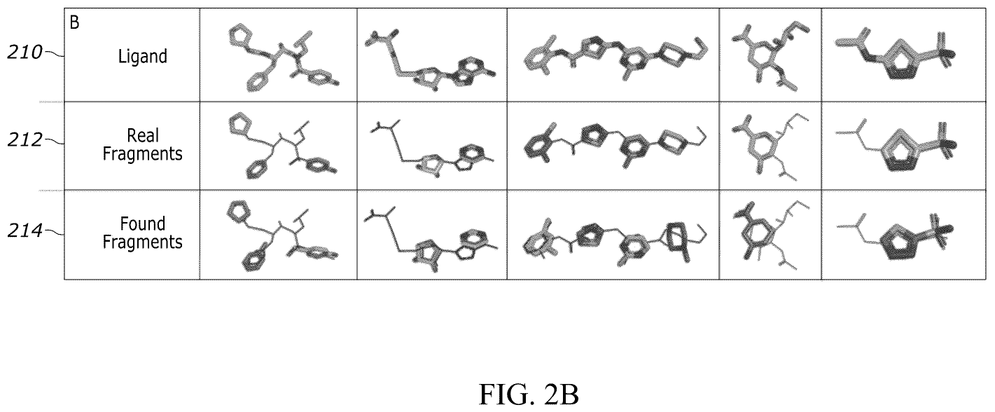

To illustrate that global embeddings of pockets contain information about the types of binding ligands, pockets bound by five structurally different ligands are selected, all-by-all pairwise similarities between pairs of their cognate binding pockets are computed, and the results are visualized using multidimensional scaling. As pockets corresponding to the same ligand should show functional and structural similarity, the embeddings of these pockets are expected to be clustered in the plane. The embeddings of the selected pockets should be grouped in 5 clusters, where each cluster corresponds to a certain ligand. The resulting distribution of points 200 is shown in FIG. 2A and clearly demonstrates that pockets binding to the same ligands are grouped together.

### 3.2 Fragment Search

The fragment search algorithm is evaluated in the same way as the ligand-search approach (Section 3.1). Instead of ligand-pocket pairs, fragment-patch pairs are considered, and candidates for each fragment's patch are searched for separately. The scoring neural network explained in Section 2.3 was retrained specifically for scoring patches of fragments. Pearson and Spearman correlation of the predicted scores are computed with Tanimoto similarity scores between fragments, and the method described herein is compared with KRIPO. KRIPO failed on 16 query patches, hence the results discussed below were obtained on 424 query patches successfully processed by all the methods. Table 2 summarizes the performance of the fragment search pipeline. It includes results obtained after the ultra-fast search stage and after the scoring step that processed patches aligned with RANSAC and our optimization-based algorithm. FIG. 2B contains several ligands 210 with their ground-truth fragments 212 and fragments 214 identified and aligned with RANSAC. In each case, fragments were manually selected from the top-10 candidates returned by the algorithm.

TABLE 2
Fragment search results.

| Method | Top-1 ↑ | Top-5 ↑ | Top-10 | Top-20 | Pearson | Spearman |
|---|---|---|---|---|---|---|
| KRIPO (Wood et al., 2012) | 0.469 | 0.724 | 0.816 | 0.873 | 0.089 | 0.098 |
| ours:search | 0.255 | 0.717 | 0.892 | 0.958 | 0.346 | 0.127 |
| ours:search + ransac + scoring | 0.245 | 0.594 | 0.715 | 0.861 | 0.119 | 0.126 |
| ours:search + optim + scoring | 0.245 | 0.479 | 0.601 | 0.776 | 0.172 | 0.136 |

### 3.3 Affinity Prediction

To assess the ability of the model to learn useful representations of protein pocket surfaces in the context of small-molecule binding, a binding affinity predictor is trained. The scenario in which a co-crystallised structure is unavailable is the most relevant, as this is the most likely scenario in practice. The approach described herein is compared to baseline methods evaluated on docked poses of ligands in the PDBbind v2016 core set and demonstrate state-of-the-art performance without requiring accurate docking as an initial step (Table 3). The approach described herein is also compare to baseline models evaluated on poses from co-crystallised structures and perform on par with the alternative methods despite not making use of pose information (Table S12 below).

TABLE 3
Binding affinity prediction performance
on PDBbind v2016 Core Set Docking Poses.

| Method | RMSE ↓ | MAE ↓ | R ↑ |
|---|---|---|---|
| SG-CNN* | 1.576 | 1.277 | 0.699 |
| 3D-CNN* | 2.558 | 2.058 | 0.537 |
| Vina* (Trott & Olson, 2009) | — | — | 0.616 |
| MM-GBSA* | — | — | 0.629 |
| FAST-midlevel* (Jones et al., 2021) | 1.874 | 1.487 | 0.702 |
| FAST-late* (Jones et al., 2021) | 1.871 | 1.498 | 0.712 |
| Ours | 1.540 | 1.248 | 0.721 |

[*] results taken from Jones et al. (2021).

### 4 Conclusion

Accurate prediction of small-molecule protein interactions remains a very challenging task for computational methods. Herein is proposed a general framework that leverages protein surface descriptors for small molecule related tasks. The novel ligand and fragment searching methods can be employed as starting points in FBDD or as initialisations to generative chemistry models to develop novel chemical matter in a principled structure-based manner. Furthermore, this approach has been developed using a surface embedding model trained for predicting protein-protein interactions. There is significant scope to improve performance by developing surface encoders explicitly trained on tasks more closely related to protein-ligand interactions. It is emphasized that properties of the surface embedding space play a crucial role in the ability to identify similar pockets based on dot product or distance similarity metrics. Therefore, an ideal protein surface encoder for this task should be trained in a way that it constrains the resulting embedding space to be Euclidean. Additionally, a binding affinity predictor is developed that is comparable in performance to existing methods without explicit consideration of pose or modelling of intramolecular interactions. Incorporating pose information is a natural extension of prior work though this framing does not retain some of the reduced pose sensitivity advantages we sought to. The components discussed herein can be combined to use a fully-differentiable affinity predictor as a scoring function to directly optimize fragment placement. The resulting set of fragments can be further merged into a single chemically relevant molecule.

### A Ligand Search Parameters

Pocket construction The radius of the neighborhood used for extracting binding pockets plays an important role in the performance of the global search. Fixing the remaining parameters different neighborhood radii (shown in Table S4) were experimented with and an optimal radius of r=2 Å was identified.

TABLE S4
Global ligand search experiments with
different neighborhood radius values.

| Radius (Å) | Top-1 ↑ | Top-5 | Top-10 | Top-20 | Top-50 |
|---|---|---|---|---|---|
| 2.0 | 0.331 | 0.508 | 0.605 | 0.774 | 0.935 |
| 3.0 | 0.306 | 0.508 | 0.581 | 0.750 | 0.911 |
| 1.0 | 0.282 | 0.516 | 0.605 | 0.782 | 0.919 |
| 10.0 | 0.282 | 0.492 | 0.613 | 0.766 | 0.887 |
| 5.0 | 0.250 | 0.460 | 0.605 | 0.790 | 0.895 |

### Global Search

To find the best way of constructing global pocket embeddings, two aggregation schemes were experimented with: simple averaging and taking the embedding vector of the point closest to the center of mass of the pocket. For similarity measures, Euclidean distance and dot product were considered. Fixing the remaining parameters, a global search experiments were performed with different aggregation and similarity functions (Table S5) identifying mean aggregation and Euclidean distance similarity as the most performant schemes.

TABLE S5
Global ligand search experiments with different
aggregation and similarity functions.

| Averaging | Similarity | Top-1 ↑ | Top-5 | Top-10 | Top-20 | Top-50 |
|---|---|---|---|---|---|---|
| Mean | Euclidean | 0.331 | 0.508 | 0.605 | 0.774 | 0.935 |
| Center | Euclidean | 0.274 | 0.548 | 0.685 | 0.815 | 0.887 |
| Mean | Dot | 0.024 | 0.024 | 0.113 | 0.274 | 0.452 |
| Center | Dot | 0.024 | 0.194 | 0.306 | 0.444 | 0.613 |

dMaSIF

The choice of the pre-trained dMaSIF model plays an important role. dMaSIF models pre-trained for three different tasks were considered: protein-protein interaction search (dMaSIF-search), protein-ligand binding affinity prediction (dMaSIF-affinity), and protein-ligand pocket classification (dMaSIF-ligand). An important dMaSIF property that was assumed should matter in this task is granularity of surfaces produced by dMaSIF. This property is controlled by two hyperparameters: subsampling and resolution. Subsampling determines the initial number of points sampled around each atom during the first step of the dMaSIF pipeline of Sverrisson 2021. Resolution controls the size of a 3D voxel that should contain not more than one point. The lower the resolution, the more detailed the resulting surface will be. Two dMaSIF-search models were considered: with subsampling 100 and resolution 1 Å, and with subsampling 150 and resolution 0.7 Å. Due to the high computational complexity, subsampling 20 and resolution 1 Å were set for dMaSIF-affinity. For the same reason, the number of training epochs differs for each dMaSIF model.

TABLE S6
Global ligand search experiments with different pre-trained dMaSIF models.

| Model | Subsampling | Resolution (A°) | Top-1 ↑ | Top-5 | Top-10 | Top-20 | Top-50 |
|---|---|---|---|---|---|---|---|
| dMaSIF-search | 150 | 0.7 | 0.387 | 0.516 | 0.629 | 0.734 | 0.871 |
| dMaSIF-search | 100 | 1.0 | 0.331 | 0.508 | 0.605 | 0.774 | 0.935 |
| dMaSIF-affinity | 20 | 1.0 | 0.266 | 0.516 | 0.653 | 0.782 | 0.871 |
| dMaSIF-ligand | 100 | 1.0 | 0.242 | 0.395 | 0.516 | 0.661 | 0.823 |

Parameters for dMaSIF models listed in Table S6 are provided in Table S7. For training the models, the code from https://github.com/FreyrS/dMaSIF was used. In case of dMaSIF-ligand and dMaSIF-affinity, the code was slightly adjusted for new purposes. dMaSIF-search models were trained on the same dataset as in Sverrisson 2021. dMaSIF-ligand was trained on the dataset that was used for training MaSIF-ligand as described by P. Gainza, F. Sverrisson, F. Monti, E. Rodol a, D. Boscaini, M. M. Bronstein, and B. E. Correia in Deciphering interaction fingerprints from protein molecular surfaces using geometric deep learning as published in Nature Methods, 17 (2): 184-192, December 2019. Dataset for dMaSIF-affinity is described in Section 2.5.

TABLE S7
Parameters of dMaSIF models used in this work.

| model | dMaSIF-search | dMaSIF-search | dMaSIF-ligand | dMaSIF-affinity |
|---|---|---|---|---|
| embedding_layer | dMaSIF | dMaSIF | dMaSIF | dMaSIF |
| resolution | 0.7 | 1.0 | 1.0 | 1.0 |
| distance | 1.05 | 1.05 | 1.05 | 1.05 |
| variance | 0.1 | 0.1 | 0.1 | 0.1 |
| sup_sampling | 150 | 100 | 100 | 10 |
| atom_dims | 6 | 6 | 6 | 6 |
| emb_dims | 16 | 16 | 16 | 32 |
| in_channels | 16 | 16 | 16 | 16 |
| orientation_units | 16 | 16 | 16 | 16 |
| post_units | 8 | 8 | 8 | 8 |
| n_layers | 3 | 3 | 3 | 1 |
| radius | 9.0 | 9.0 | 9.0 | 9.0 |
| epochs | 33 | 50 | 21 | 108 |

### B Alignment

### B.1 Alignment Experiment

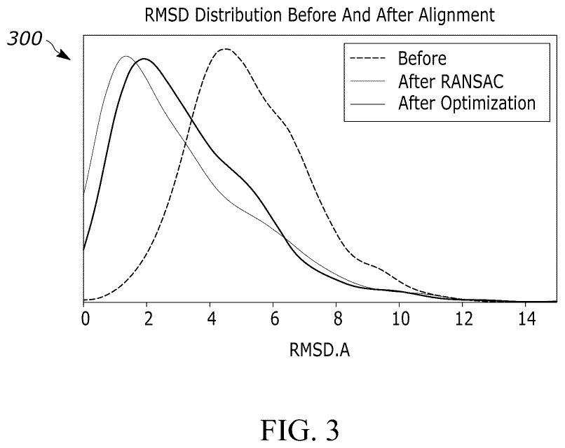

To perform alignment, a subset of 460 pocket pairs are selected from the training set of the scoring neural network (section 2.3) and aligned with RANSAC or the optimization-based approach described in section 2.2. For RANSAC, three different modifications are considered: RANSAC, RANSAC followed by point-to-point ICP, and RANSAC followed by point-to-plane ICP. For the optimization-based method, three objectives are considered based on different underlying similarity functions: Euclidean distance, dot product, and cosine distance. To measure the quality of aligned pairs, the root-mean-square deviation (RMSD) is computed between ligand atoms that are transformed according to the corresponding pocket alignment. The initial RMSD between atoms of ligands (i.e. before alignment), and final RMSD computed on transformed ligands is reported in Table S8. The fraction of pairs for which alignment improved RMSD between atoms of ligands are also provided. For each method reported in Table S8, the best set of hyperparameters is chosen. For details, see Appendix B.2. FIG. 3 shows the RMSD distribution 300 over all pairs before and after alignment. The distribution of errors on pockets aligned by RANSAC is shifted towards zero most, which makes this method the most preferable. On average, alignment of one pocket pair takes 0.17 seconds for RANSAC and 2.33 seconds for our optimization-based algorithm.

TABLE S8
Alignment results.

| Alignment Method | Initial RMSD (Å) | Final RMSD (Å) | Improvement Rate |
|---|---|---|---|
| RANSAC | 5.281 | 3.174 | 0.726 |
| RANSAC + PointToPoint | 5.281 | 3.082 | 0.772 |
| RANSAC + PointToPlane | 5.281 | 3.915 | 0.689 |
| Optimization, Euclidean | 5.281 | 4.332 | 0.667 |
| Optimization, Dot | 5.281 | 4.325 | 0.663 |
| Optimization, Cosine | 5.281 | 3.354 | 0.770 |

### B.2 Alignment Parameters

In order to align pockets with RANSAC and ICP, the Open3D implementations of these algorithms were used. Different distance threshold values (between 1 Å and 10 Å) and different combinations of RANSAC and ICP were considered: RANSAC, RANSAC+PointToPoint and RANSAC+PointToPlane. The final parameter set with the best performance is reported in Table S9.

TABLE S9
Best configuration of RASNAC + ICP parameters.

| ICP | distance_threshold | ransac_n | max iter | max validation |
|---|---|---|---|---|
| PointToPoint | 2.0 | 3 | 100000 | 10000 |

For the optimization-based alignment algorithm, three different similarity metrics (Euclidean distance, dot product, and cosine) were considered and different values for the variance σ∈{0.5, 1.0, 2.0, 5.0, 10.0} of Gaussian kernels (1). β∈{0.0, 0.25, 0.5, 0.75, 1.0} was also varied in order to study the contribution of embeddings- and normals-related terms (2) to the total objective. Before alignment, point clouds were matched based on their centers of mass. Translation parameter and rotation parameters were optimized with different learning rates, lrₜ and lr_{R} correspondingly. Once the initial matching by centers of mass is performed, translation should not differ much from zero furthermore. Hence, the learning rate for the translation parameter was usually several orders of magnitude lower than learning rate for rotation parameters. In all experiments, 1,000 steps of optimization were performed. The best parameter configurations for each similarity function along with final RMSD results are provided in Table S10.

TABLE S10
Best parameter configurations of the optimization-based
alignment algorithm with different similarity functions.

| Similarity | σ | β | lrₜ | lr_{B} | Final RMSD |
|---|---|---|---|---|---|
| ↑ Euclidean | 10.0 | 0.75 | 0.0001 | 0.1 | 4.332 |
| Dot | 10.0 | 0.75 | 0.0001 | 0.1 | 4.325 |
| Cosine | 5.0 | 1.0 | 0.0001 | 1.0 | 3.354 |

### C Scoring Neural Network

The neural network was trained to solve the binary classification problem using cross-entropy loss. The network was trained for 100 epochs with batch size 128 using Adam optimizer with learning rate 10⁻⁴.

### D Fragments

Examples of fragments 400 retrieved using BRICS are shown in FIG. 4. Occurrences of fragments in the data are denoted beneath fragment representations.

### E Binding Affinity Prediction

### E.1 Hyperparameters

TABLE S11
Hyperparameters considered for binding affinity predictor.

| Component | Hyperparameter | Values |
|---|---|---|
| Surface Encoder | embedding_layer | dMaSIF |
| | resolution | 1.0 |
| | distance | 1.05 |
| | variance | 0.1 |
| | sup_sampling | 10, 20 |
| | atom_dims | 6 |
| | emb_dims | 32 |
| | in_channels | 16 |
| | orientation_units | 16 |
| | post_units | 8 |
| | n_layers | 1, 3 |
| | radius | 9.0, 15.0 |
| Ligand Encoder | Layers | [GCN, GCN, GCN, Linear] |
| | Dims | [32, 32, 32, 32], [128, 128, 128, 32] |
| Decoder | Dims | [128, 128, 128, 1], [1024, 512, 128, 1], [1024, 1024, 1024, 1] |
| optimiser | Batchsize | 128 |
| | dropout | 0.3 |
| | lr | 0.00001, 0.0001, 0.001 |
| | Adam | |

E.2 Comparison on Poses from Co-Crystallised Structures

TABLE S12
Binding affinity prediction performance
on PDBbind v2016 Core Set Docking Poses.

| Method | RMSE ↓ | MAE ↓ | R ↑ |
|---|---|---|---|
| SG-CNN (R)* | 1.650 | 1.321 | 0.666 |
| SG-CNN (G)* | 1.508 | 1.277 | 0.699 |
| SG-CNN (R + G)* | 1.375 | 1.084 | 0.782 |
| 3D-CNN (R)* | 1.501 | 1.164 | 0.723 |
| 3D-CNN (G)* | 1.655 | 1.294 | 0.649 |
| 3D-CNN (R + G)* | 1.688 | 1.334 | 0.677 |
| Pafnucy* | 1.42 | 1.13 | 0.78 |
| (Stepniewska-Dziubinska et al., 2018) | | | |
| Kdeep* (Jime'nez et al., 2018) | 1.27 | — | 0.82 |
| FAST-midlevel* (Jones et al., 2021) | 1.308 | 1.019 | 0.810 |
| FAST-late* (Jones et al., 2021) | 1.326 | 1.044 | 0.808 |
| Ours | 1.540 | 1.248 | 0.721 |

*results taken from Jones et al. (2021). (R) and (G) denote models trained on the refined and general sets of PDBbind v2016.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

Embodiments of the invention may be implemented on a computer system. The computer system may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system may comprise any circuit or combination of circuits. In one embodiment, the computer system may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), or any other type of processor or processing circuit. Other types of circuits that may be included in the computer system may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system may also include a display device, one or more speakers, and a keyboard and/or controller, which can include a mouse, trackball, touch screen, voice-recognition device, or any other device that permits a system user to input information into and receive information from the computer system.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine-readable carrier.

Other embodiments comprise the computer program for performing one of the methods described herein, stored on a machine-readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Embodiments may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, metadata and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g., a classification algorithm, a regression algorithm or a similarity learning algorithm. Classification algorithms may be used when the outputs are restricted to a limited set of values (categorical variables), i.e., the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g., of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e., to achieve a desired output for a given input.

Alternatively, the machine-learning model may be a support vector machine, a random forest model or a gradient boosting model. Support vector machines (i.e., support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

The following aspects of the disclosure are exemplary only and not intended to limit the scope of the disclosure.
1. A computer-implemented method for predicting small-molecule protein interactions, comprising: providing a library set of binding specifications based at least in part on pockets of proteins, each binding specification of the library set corresponding to a binding candidate; providing a query set of binding specifications; aligning the binding specifications of the library set with the binding specifications of the query set; and scoring the aligned binding specifications based at least in part on using a scoring neural network, the scoring neural network being pre-trained for ranking the aligned binding specifications.
2. The method of aspect 1, wherein the binding specifications are ligand binding pockets of the proteins or patches, the patches corresponding to smaller regions of ligand binding pockets.
3. The method of any one of the preceding aspects, wherein the binding candidates are ligands or fragments, the fragments being decomposed based at least in part on ligands.
4. The method of any one of the preceding aspects, wherein the step of aligning results in aligned pairs of the binding specifications each pair forming a match, the scoring ranking the matches to identify binding candidates that are most likely to bind to the proteins.
5. The method of any one of the preceding aspects, wherein the binding specifications of the library set and/or of the query set, particularly protein pockets, are generated based at least in part on a surface encoding, the surface encoding comprising at least: generating a surface representation of each of the proteins; and computing embeddings for each point on the surface representation.
6. The method of aspect 5, wherein the surface encoding comprises further:
selecting the closest point on the surface representation for each of the binding candidates, particularly ligands; and generating at least one pocket embedding based at least in part on the selected points.
7. The method of aspect 5 or 6, wherein the surface encoding is carried out at least partially using a trained surface encoding model, particularly for computing the embeddings.
8. The method of any one of the preceding aspects, wherein for each binding specification of the query set, a search for the binding specifications of the library set is carried out based at least in part on a similarity with the binding specification of the query set, the search resulting in a shortlist set of the binding candidates to which the binding specifications of the library set found by the search correspond, the binding candidates of the shortlist set being provided as potential candidates for the binding specification of the query set.
9. The method of aspect 8, wherein the shortlist set is limited to at most 100 or at most 70 or at most 50 potential candidates.
10. The method of aspect 8 or 9, wherein the search is carried out based at least in part on at least one similarity function, the at least one similarity function being at least one of the following: Euclidean distance, dot product, or cosine.
11. The method of any one of the preceding aspects, wherein the step of aligning comprises carrying out algorithms based at least in part on: a Random Sample Consensus (RANSAC) followed by point-to-point Iterative Closest Point (ICP).
12. The method of any one of the preceding aspects, wherein the step of aligning comprises an optimization-based alignment.
13. A method for training a scoring neural network for ranking aligned binding specifications, comprising: obtaining training data, the training data comprising a library set of binding specifications based at least in part on pockets of proteins, each binding specification of the library set corresponding to a binding candidate, the training data further comprising a query set of binding specifications, training the scoring neural network based at least in part on the training data.
14. The method of aspect 13, further comprising: creating positive training examples based at least in part on the obtained training data by selecting pairs of the binding specifications that correspond to the same binding candidates; creating negative training examples based at least in part on the obtained training data by selecting pairs of the binding specifications that correspond to different binding candidates.
15. The method of aspect 14, further comprising: splitting the positive training examples and negative training examples randomly into training and validation sets.
16. The method of any one of aspects 13 to 15, further comprising: carrying out the method of any one of aspects 1 to 12, the step of scoring being carried out based at least in part on the scoring neural network trained in accordance with the method of any one of aspects 13 to 15.
17. A computer-readable medium storing a scoring neural network configured to, when implemented by a processor, rank aligned binding specifications, the scoring neural network trained with an algorithm in accordance with the method of any one of aspects 13 to 16.
18. A data processing apparatus comprising: a processor; a computer memory communicatively coupled to the processor; and computing instructions stored in the memory that when executed by the processor, cause the processor to implement an algorithm according to the method of any one of aspects 1 to 16.
19. A computer-readable medium storing a computer program comprising instructions which, when executed by a computer, cause the computer to execute an algorithm in accordance with the method of any one of aspects 1 to 16.

1. A computer-implemented method for predicting small-molecule protein interactions, comprising:
providing a library set of binding specifications based at least in part on pockets of proteins, each binding specification of the library set corresponding to a binding candidate;
providing a query set of binding specifications;
aligning the binding specifications of the library set with the binding specifications of the query set; and
scoring the aligned binding specifications based at least in part on using a scoring neural network, the scoring neural network being pre-trained for ranking the aligned binding specifications.

2. The method of claim 1, wherein the binding specifications are ligand binding pockets of the proteins or patches, the patches corresponding to smaller regions of ligand binding pockets.

3. The method of claim 1, wherein the binding candidates are ligands or fragments, the fragments being decomposed based at least in part on ligands.

4. The method of claim 1, wherein the step of aligning results in aligned pairs of the binding specifications each pair forming a match, the scoring ranking the matches to identify binding candidates that are most likely to bind to the proteins.

5. The method of claim 1, wherein the binding specifications of the library set and/or of the query set, particularly protein pockets, are generated based at least in part on a surface encoding, the surface encoding comprising at least:
generating a surface representation of each of the proteins; and
computing embeddings for each point on the surface representation.

6. The method of claim 5, wherein the surface encoding comprises further:
selecting the closest point on the surface representation for each of the binding candidates, particularly ligands; and
generating at least one pocket embedding based at least in part on the selected points.

7. The method of claim 5, wherein the surface encoding is carried out at least partially using a trained surface encoding model, particularly for computing the embeddings.

8. The method of claim 1, wherein for each binding specification of the query set, a search for the binding specifications of the library set is carried out based at least in part on a similarity with the binding specification of the query set, the search resulting in a shortlist set of the binding candidates to which the binding specifications of the library set found by the search correspond, the binding candidates of the shortlist set being provided as potential candidates for the binding specification of the query set.

9. The method of claim 8, wherein the shortlist set is limited to at most 100 or at most 70 or at most 50 potential candidates.

10. The method of claim 8 or, wherein the search is carried out based at least in part on at least one similarity function, the at least one similarity function being at least one of the following: Euclidean distance, dot product, or cosine.

11. The method of claim 1, wherein the step of aligning comprises carrying out algorithms based at least in part on: a Random Sample Consensus (RANSAC) followed by point-to-point Iterative Closest Point (ICP).

12. The method of claim 1, wherein the step of aligning comprises an optimization-based alignment.

13. A method for training a scoring neural network for ranking aligned binding specifications, comprising:
obtaining training data, the training data comprising a library set of binding specifications based at least in part on pockets of proteins, each binding specification of the library set corresponding to a binding candidate, the training data further comprising a query set of binding specifications,
training the scoring neural network based at least in part on the training data.

14. The method of claim 13, further comprising:
creating positive training examples based at least in part on the obtained training data by selecting pairs of the binding specifications that correspond to the same binding candidates;
creating negative training examples based at least in part on the obtained training data by selecting pairs of the binding specifications that correspond to different binding candidates.

15. The method of claim 14, further comprising:
splitting the positive training examples and negative training examples randomly into training and validation sets.

16. The method of claim 13, further comprising:
providing a library set of binding specifications based at least in part on pockets of proteins, each binding specification of the library set corresponding to a binding candidate;
providing a query set of binding specifications;
aligning the binding specifications of the library set with the binding specifications of the query set; and
scoring the aligned binding specifications based at least in part on using the scoring neural network as trained.

17. (canceled)

18. A data processing apparatus comprising:
a processor;
a computer memory communicatively coupled to the processor; and
computing instructions stored in the memory that when executed by the processor, cause the processor to:
provide a library set of binding specifications based at least in part on pockets of proteins, each binding specification of the library set corresponding to a binding candidate;
provide a query set of binding specifications;
align the binding specifications of the library set with the binding specifications of the query set; and
score the aligned binding specifications based at least in part on using a scoring neural network, the scoring neural network being pre-trained for ranking the aligned binding specifications.

19. (canceled)
